(19) United States
(12) Patent Application Publication
(10) Pub. No.: US 2023/0263642 A1
(43) Pub. Date: Aug. 24, 2023

(54) IMPLANTS AND PRODUCTION METHODS OF PRODUCTS FOR BONE REGENERATION

(71) Applicant: UNIVERSIDAD DE LOS ANDES, Bogota (CO)

(72) Inventors: Anibal Faruk ABEDRABBO HAZBUN, Bogota (CO); Oscar Javier BENAVIDES ORTIZ, Bogota (CO); Rene Santiago BERNAL BAEZ, Bogota (CO); Hector Mauricio FRANCO CARRILLO, Chia (CO); Paula Alejandra GOMEZ VILLALOBOS, Bogota (CO); Jaime Andres MORENO RAMOS, Tunja (CO); Fabio Arturo ROJAS MORA, Conjunto El Nogal Tenjo (CO)

(21) Appl. No.: 18/006,432

(22) PCT Filed: Jul. 23, 2021
(86) PCT No.: PCT/IB2021/056682

§ 371 Date: Apr. 6, 2023

(30) Foreign Application Priority Data
Jul. 23, 2020 (CO) NC2020/0009088

(51) Int. Cl. A61F 2/46; A61F 2/28; B33Y 40/20

(52) U.S. Cl. CPC A61F 2/4644; A61F 2/28; B33Y 40/20; A61F 2002/2835; A61F 2002/4645; B33Y 80/00

(57) ABSTRACT

A method for the execution of implants, surface finishes of implants the manufacture of a particulated bone material, and with the particulated bone material obtained, a method for the manufacture of a bone-biopolymer filament may be performed, to obtain a bone-biopolymer filament. The filament may be used, for example, in a method for the manufacture of implants by 3D-printing. A method for the manufacture of bone implants by machining; once a bone implant has been obtained by the method of manufacture of implants by 3D-printing or the method of manufacture of bone implants by machining, it is subjected to a texturisation to enable the osseointegration thereof.

### FIELD OF THE INVENTION

The present invention relates to bone implants for repair, replacement, and/or augmentation of different portions of animal or human skeletal systems, and methods for manufacturing bone implants.

### DESCRIPTION OF THE PRIOR ART

Currently, and regarding the field of prosthetic implants, different types of implants are used to replace bone tissue. Many of these implants are made from non-bone materials, such as stainless steel, titanium, and some polymers. However, the use of some of the previously mentioned non-osseous materials sometimes generate rejection by the body of users, which is why different types of implants are required to implement bone as raw material, in such a way that the body of a patient does not generate rejection.

However, the manufacture of such bone-containing implants is difficult because bones used in the manufacture of implants do not have standard dimensions or ratios allowing them to be molded by conventional manufacturing methods. Therefore, different manufacturing methods are required to obtain bone implants with specific shapes, wherein said implants can fulfill the function of improving the bone reconstruction process of the recipient subject.

Therefore, the prior art discloses documents that teach different types of bone implants, and manufacturing methods, such as documents U.S. Pat. No. 6,458,158B1, WO2000066011A1, U.S. Pat. Nos. 5,053,049A, 4,627,853A, and ES2209988T3.

Document U.S. Pat. No. 6,458,158B1 discloses a bone graft to be implanted and manufacturing methods thereof. The bone graft can be formed from cortical bone. Among the processes indicated for manufacturing the graft, turning, and milling are implemented. Likewise, it is stated that the graft may comprise one or several textures on its surface depending on the place and the function that it will fulfill. In one embodiment, the graft is a pin with a rough surface.

On the other hand, document WO2000066011A1 discloses a xenograft, specifically a screw formed from cortical bone (of non-human origin) to be used as an implant. The cortical bone is machined to obtain the screw, and the indicated means of manufacture are a lathe, a Swiss milling machine, a CNC, a thread turning machine, or a similar device to machine the screw out of the cortical bone to specific dimensions.

On the other hand, document U.S. Pat. No. 5,053,049A discloses processes for manufacturing prostheses from bone according to the desired shape. Among the processes, machining, demineralization, and tanning are stated. The machining processes include milling, drilling, saw-cutting, among others. Likewise, it is stated that the bone can be ground and/or pulverized. The pulverization provides a vehicle which may be biologically compatible, and this product can be machined by the aforesaid methods. Finally, the machined part is treated to increase porosity and for cleaning.

Document U.S. Pat. No. 4,627,853A discloses prostheses for cartilage replacement, which can be obtained from demineralized bone machining. The machining is carried out until the desired shape and size are obtained. Any of the conventional machining processes can be used for manufacturing, such as turning, drilling, milling, saw-cutting, among others. The machined piece is treated in order to increase porosity, since osteoconductivity of an implant is directly related to its ability to be vascularized within the recipient body and is estimated through the porous holes it contains.

Finally, document ES2209988T3 discloses a resorbable material for bone replacement and regeneration (augmentation material) based on porous tricalcium phosphate (-TCP). This disclosure further indicates that porosity increases the specific surface area and with it the resorption capacity which in turn stimulates bone regeneration activity by osteoblasts only in young patients. Likewise, this process reduces the mechanical resistance and increases the tendency to particular decomposition. However, the document states that porosity must be controlled, since microporosity (pores less than 20 microns, in this document) leads to a capillary suction effect of liquids around the implant, and bone structures and blood vessels do not manage to penetrate the areas where the liquid is sucked into the micropores, so necrosis of cells and sucked liquids may occur. On the other hand, macro-porosity (pores with radius greater than 20 micrometers) allows the penetration of bone into the pores.

Therefore, although the cited documents refer to different methods and materials for bone regeneration, said documents do not disclose how to improve osseointegration in implants, nor how to prevent a user's body from rejecting said implants. Additionally, said documents do not reveal the detailed manufacturing conditions to obtain the implants industrially.

### BRIEF DESCRIPTION OF THE INVENTION

The present invention relates to methods for making implants, and to the surface finishes of said implants. One of the methods of the present invention is a method for manufacturing a particulate bone material (100) which allows bone powder production. Additionally, with the particulate bone material that is obtained, it is possible to carry out a method for manufacturing a filament based on bone and biopolymer to obtain a bone-biopolymer filament. Said filament can be used, for example, in a 3D printing implant manufacturing method that allows the production of an implant for bone reconstruction based on 3D printing techniques.

In addition to the foregoing methods, the present invention also refers to a method for manufacturing bone implants by machining, which allows machining of the implants avoiding bone fracture.

Therefore, once a bone implant is obtained, either by the implant manufacturing method known as 3D printing or the bone implant manufacturing method by machining, said implant is texturized, which accelerates the osseointegration thereof, reducing the possibility of implant rejection by the patient.

### BRIEF DESCRIPTION OF THE FIGURES

FIG. 1 illustrates a block diagram showing the interaction of different methods to obtain textured bone implants.

FIG. 2 illustrates a block diagram related to the pre-treatment and cleaning of a bone for later use in the manufacture of bone implants.

FIG. 3 illustrates a block diagram related to a method for obtaining a particulate bone material.

FIG. 4 illustrates a block diagram related to a method for manufacturing filaments, including bone material and biopolymers.

FIG. 5 illustrates a block diagram related to a method for manufacturing bone implants by machining.

FIG. 6A illustrates two views of a machined manufactured bone implant which has a thread.

FIG. 6B illustrates a view of a machined manufactured bone implant which has a thread.

FIG. 7A illustrates a view of a dog x-ray, wherein a bone implant implanted in a bone after surgery is shown.

FIG. 7B illustrates a view of a dog x-ray, showing the bone implant implanted in a bone of FIG. 7A, after 30 days.

FIG. 7C illustrates a view of a dog x-ray, wherein a bone implant implanted in a bone is shown.

FIG. 8A is a picture of the microstructure of a bone implant surface.

FIG. 8B is a picture of the microstructure of a bone implant surface.

FIG. 8C is a picture of the microstructure of a bone implant surface.

FIG. 9A is a picture of the microstructure of a bone implant surface.

FIG. 9B is a picture of the microstructure of a bone implant surface.

FIG. 9C is a picture of the microstructure of a bone implant surface.

FIG. 10A is a photo of a 3D printed and subsequently machined bone implant.

FIG. 10B is a photo of a 3D printed and subsequently machined bone implant.

FIG. 11A is a photo of a machined bone implant.

FIG. 11B is a photo of the bone implant of FIG. 11A implanted in a dog bone.

CHART 1 illustrates a diagram of the cutting angles of a cutting tool.

### DETAILED DESCRIPTION

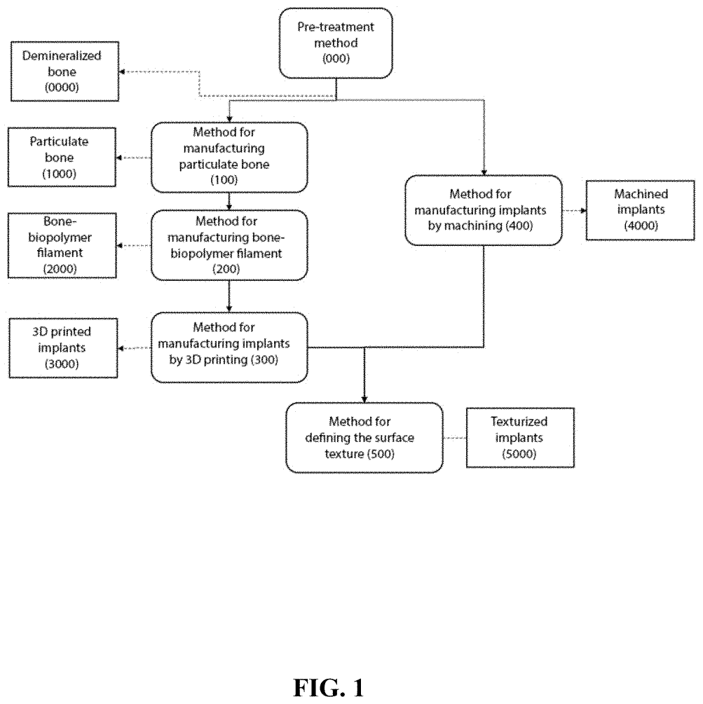

Referring to FIG. 1, the present invention refers to different types of methods that allow different types of bone implants to be made, which can be used as replacement material, bone filler material or support material, allowing the reconstruction of a bone fracture, by means of an implant that reconstructs the fracture, being absorbed and regenerated within the patient. Additionally, the present invention also refers to the different types of treatments that implants can have, which allow their properties to be improved, which allows the osseointegration and regeneration of a lesion in a patient to whom the implant is placed, to be performed in less time and avoiding the need for a second surgery to remove the implants.

Once a bone matrix material is obtained to make an implant, the present invention refers to different types of methods for manufacturing implants. Specifically, the methods are method for manufacturing particulate bone (100), method for manufacturing bone-biopolymer filament (200), method for manufacturing implants by 3D printing (300), and method for manufacturing implants by machining (400).

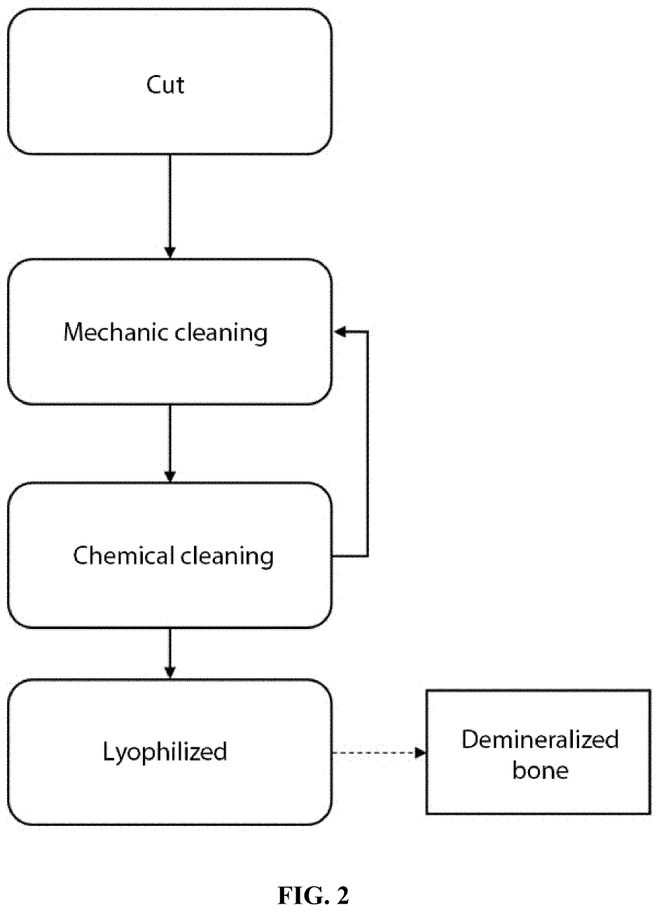

Referring to FIG. 2, and an embodiment of the invention, a pre-treatment method (000) is performed on a bone material, in this case, a bone or a plurality of bones that will be used to manufacture a bone implant. From said pre-treatment method (000), a lyophilized bone (0000) is obtained that can be used as an input to manufacture bone implants.

After said pre-treatment method (000), and continuing with FIG. 2, different types of methods can be performed to obtain different bone implants. One of them is a method for manufacturing a particulate bone material (100), which allows obtaining particulate bone material (1000). In addition to said method, and with the particulate bone material (1000) obtained, a method for manufacturing a bone-biopolymer filament (200) can be carried out in order to obtain a bone-biopolymer filament (2000). Where said bone-biopolymer filament (2000) can be used, e.g., in a method for manufacturing implants by 3D printing (300). Where said manufacturing method for implants by means of 3D printing (300) allows obtaining an implant for bone reconstruction printed in 3D (3000).

In addition to the aforementioned methods, the present invention also refers to using freeze-dried bone (0000) as a raw material in a method for manufacturing bone implants by machining (400). Therefore, once a bone implant is obtained, either obtained by the method for manufacturing implants by 3D printing (300) or the method for manufacturing bone implants by machining (400), a texturing method is performed on said implant (500) which allows the best osseointegration thereof, decreasing the probability of implant rejection by the patient.

Specifically, and regarding the pre-treatment method (000), it consists of the following: first a bone is provided, wherein said bone may have a cortical portion (referred to as compact bone) and a trabecular portion (referred to as spongy bone). Then, a cut is made to the bone, where the epiphyses are removed, i.e., the bone ends with the highest cancellous or trabecular bone content and then the bone is cut longitudinally along the rough line of the bone, separating it into two sections. Once this cut is made, a mechanical cleaning is implemented, wherein soft tissues are removed, leaving only the cortical bone. This can be carried out by scraping with a blade or other objects that have a sharp section, or otherwise with abrasive objects.

After mechanical cleaning, the bone is submerged in cold water, which acts as a physical denaturant against the protein contained in the blood, facilitating its removal. Next, a chemical denaturant is applied, which can be a detergent, immersing the bone in a soapy solution for a period of at least 24 hours to break the chains of bacteria attached to the bone walls. And, then, the bone is immersed in alcohol, which allows the elimination of some microbial and bacterial organisms and disables the action of other microorganisms. This process should preferably be carried out with ethyl alcohol for a period of at least 24 hours.

The mechanical and chemical cleaning steps can be performed as many times as necessary, until a completely clean bone is obtained, i.e., without soft tissue or traces of blood. Once the steps of bone cutting, mechanical cleaning, and chemical cleaning have been carried out, the bone is lyophilized, i.e., the bone is frozen and all the liquids and organic materials that the already treated bone may contain inside are extracted by vacuum pressure.

It should be taken into account that carrying out the pre-treatment steps mentioned above allows obtaining a bone that serves as an input in the manufacture of implants and tissues, considering that soft tissues are removed and the bone is cleaned, leaving as a result a portion of sterilized bone.

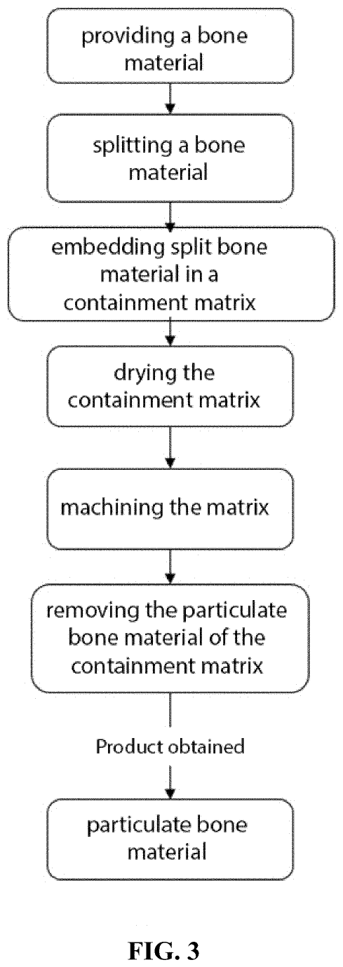

Regarding FIG. 3, and in relation to the manufacturing method for a particulate bone material (100), this comprises the following steps:
a) providing a bone material;
b) splitting a bone material into fragments;
c) embedding the split bone material in a soluble containment matrix;
d) drying the soluble containment matrix until a solidified containment matrix is obtained;
e) machining the solidified containment matrix by means of a machining process; and
f) removing the particulate bone material (1000) from the containment matrix.

Specifically and with respect to step a) of the method for manufacturing a particulate bone material (100), the bone material that is provided can be cortical lyophilized bone. On the other hand, regarding step b) of splitting the bone material, said bone material can be cut into pieces, preferably cuts 3 cm to 5 cm long, wherein said pieces can have a cylinder shape cut longitudinally. The fact that the bone material is cut as mentioned above allows the cut portions to be embedded in the containment matrix.

For the understanding of the present invention, bone material shall be understood as any material derived from animal bone or human bone. Additionally, for the understanding of the present invention, a containment matrix will be understood as a substance in a liquid state, which allows different materials to be agglomerated and subsequently solidified. An example of a containment matrix may be sugars, caramel, pure sucralose, or melted sucralose. Where said sugar is melted by raising its temperature by means of any heating method, at a temperature between 100° C. to 160° C., preferably, between 120° C. to 140° C. The fact that the containment matrix is made of sucralose, facilitates the removal of its content adhered to a bone, since it can be removed by immersing a bone with sucralose in water at temperatures below 30°, in addition to the non-toxic nature of sucralose.

Considering the foregoing, the step of embedding the split bone material in a containment matrix that is initially soluble and then solidifies, refers to embedding the pieces of bone material from step b), in a soluble matter, such as melted sugar. Where, said sugar is subsequently solidified with the pieces of bone material, thus obtaining a plurality of pieces of bone material embedded in a material with a degree of hardness, in such a way that it can be manipulated by a user and machining.

Said containment matrix can be in a mold, wherein the shape of the mold is selected from the group made up of cylinders, cubes, pyramids, prisms, equivalent shapes known to a person ordinarily skilled in the art, or a combination of the above. This allows the containment matrix to have the desired shape when it solidifies. Additionally, the mold material can be a polymeric material, or materials that have a low coefficient of friction in relation to the containment matrix, allowing said matrix to be removed from the mold without damage. Optionally, Mylar paper is used inside the mold to facilitate demolding of the containment matrix.

In one embodiment of the invention, the mold in which the containment matrix is located is cylindrical, and the material of said mold can be made of a polymer. This allows the containment matrix to be placed in a lathe when it hardens and said matrix cannot be removed from the mold without suffering damage. Additionally, and to facilitate demolding of the solidified matrix, said mold can be split into two pieces and contain clamping rings to keep it sealed while the material is poured, and the matrix solidifies. When the mold has an axis as mentioned above, said axis can be knurled to maximize the grip with the containment matrix when it is formed by sugar.

Once the containment matrix solidifies, it is removed from the mold, obtaining a structure that comprises bone material together with the solidified containment matrix, which can be machined by a user. Using a containment matrix and taking into account that the bone has a high variability in its dimensions and it is not possible to carry out a clamping for a standard machining (e.g., bone clamping methodologies), by encapsulating the bone in a defined geometry, allows obtaining a standard shape that also allows the bone to be clamped in a lathe or other machining machine. The foregoing, without any risk of coming loose and regardless of the shape and size of bone material pieces.

When the containment matrix solidifies, a solidified containment matrix is obtained. Referring to step d), the solidified containment matrix is machined by means of a machining process. Optionally, said machining process is a turning wherein the solidified containment matrix containing the bone material has an axis which is held in the mandrel of the lathe where the turning is carried out, in order to be able to crush the solidified containment matrix. On the other hand, the other end of the solidified containment matrix is held by the mobile point of the lathe. Additionally, a machined chip collector container is located on the lathe bed, which completely covers the solidified structure. This allows the collection of all the material coming after the turning process is finished, which corresponds to particulate bone material (1000) with remains of the containment matrix.

Additionally, said turning can be carried out using a cutting tool corresponding to a 12% Co high-speed steel turning tool, which can be sharpened with an alumina stone, to achieve the required angles in the tool. Said alumina stone has a lower hardness compared to other abrasives used to sharpen turning tool (e.g., carbide, diamond, CBN), which allows obtaining the edge of the cutting tool in such a way that the edge degree of the cutting tool can be precisely controlled so that the turned part is not damaged.

The parameters of such turning can be as follows:
a cutting tool feed of between 0.01 mm/rev, up to 0.06 mm/rev, preferably between 0.02 mm/rev and 0.03 mm/rev;
a machining speed of between 3 m/min and 4 m/min, preferably between 3.4 m/min and 3.5 m/min;
a rake angle between 3° and 8°, preferably between 4° and 5°, clearance angle, between 5° and 10°, and main steering angle between 50° and 65°; and
a cutting depth between 0.02 mm and 0.05 mm. These machining parameters are required to obtain machined particles with form factor (FF) between 0.5 and 1.0.

On the other hand, and with respect to step e) of the method corresponding to removing the particulate bone material from the containment matrix, the remains of the containment matrix are removed, dissolving said matrix in a solvent, which allows washing the particulate bone material. Optionally, said solvent can be water with a temperature between 20° C. and 40° C., preferably between 20° C. and 30° C. Said particulate bone material (1000) resulting from machining with remains of the containment matrix, is immersed in the solvent between 1 and 5 hours, preferably 2 to 3 hours. Additionally, the solvent must be agitated with the particulate bone material (1000) at different time intervals, which reduces the time for washing the containment matrix. Said agitation can be carried out during intervals between 20 and 30 minutes.

In addition, the solvent can be changed for different periods of time, which allows for improved washing between the particulate bone material (1000) and the containment matrix. Said solvent change time periods can be between 30 minutes and 1 hour. If it is not constantly agitated, the immersion time of the bone with the containment matrix when it is caramel will be around 8 to 24 hours, wherein it is preferable to change the solvent every hour to avoid impregnation of the caramel inside the bone. Said change of solvent is made because when the solvent comes into contact with the caramel, a supersaturated caramel substance is produced, and if this is not changed, washing does not occur, i.e., the caramel does not keep coming out of the bone, remaining on the surroundings, permeating the porosities of the bone.

Once the washing between the containment matrix and the particulate bone material (1000) is carried out, these can be separated from the solvent by sieving to eliminate the fluid from the particulate bone material (1000). Then, the particulate bone material (1000) is left to dry between 6 to 12 hours at room temperature, preferably 8 to 11 hours.

Additionally, the particulate bone material (1000) can be passed through a high energy mill, e.g., “Simple Vibrating Mill TI-100” for 1 to 2 hours, which makes it possible to reduce the particle size in case it is very large after machining. Additionally, the above allows obtaining an average size of 96.2 mm with a deviation of 14 mm and a form factor (FF) of 0.46, within approximate deviation of 0.16. Once the particulate bone material (1000) is dried and separated, it remains with diameters between 50 μm and 250 μm, and a form factor (FF) between 0.5 and 1, preferably 0.6. Optionally, bone material can be sieved to provide bone particle sizes with diameters between 90 mm and 120 mm. Additionally, in a further step said particle size can also be obtained by passing said particulate bone material through a mill until the desired size is obtained. In order to understand the present invention, form factor FF is defined as the fraction between the smaller diameter circumscribed in the grain and the larger diameter therein.

Moreover, the parameters of the aforementioned machining allow obtaining particles with a form factor (FF) close to 0.52, which allows said particles to be implemented in filament 3D printing machines to make prints with them. While traditional machining and traditional grinding processes deliver particle shapes that do not meet the typical requirements of a filament 3D printing machine, about 140 mm maximum size.

Once the particulate bone material is obtained according to the method for manufacturing a particulate bone material (100), it can be used as a bone graft for filling in orthopedic and dental injuries. Additionally, the particulate bone material serves as a raw material for bone-polymer composite matrices, as a bone graft for filling in orthopedic and dental injuries and can also be provided as a raw material for the manufacture of bone filaments for 3D printing by extrusion.

One of the technical effects of the particulate bone material having the aforementioned features, particle diameters between 50 μm and 250 μm, and a form factor (FF) between 0.5 and 1.0, preferably 0.6. The foregoing allows the particle size to be in the desired range improving the homogenization of bone-polymer composite matrices and also, when using bone-polymer filaments in a 3D printer, preventing the extruder or nozzle from clogging.

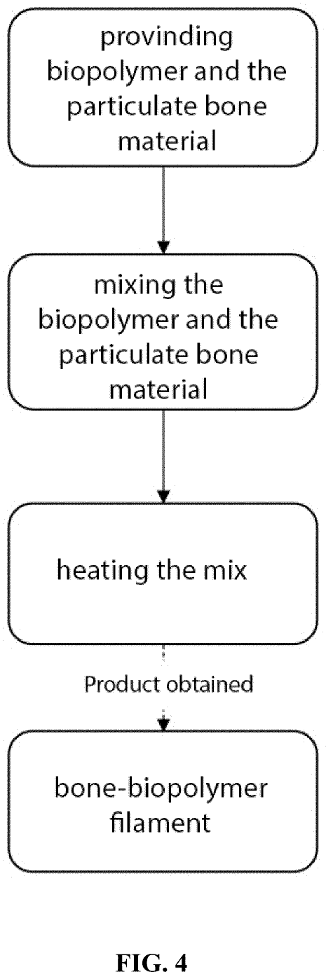

On the other hand, and referring to FIG. 4, the present invention also relates to a method for manufacturing a filament, preferably to a method for manufacturing a bone-polymer filament (200) to make bone implants. Wherein said method comprises:
a) providing biopolymer and the particulate bone material in a container;
b) mixing the biopolymer and the particulate bone material from step a);
c) heating the mixture of step c) to a temperature between 160° C. to 200° C.; and
d) extruding the mixture obtained to form the filament.

Wherein, said mixing is carried out for a period between 2 minutes to 8 minutes.

Referring to step a), the particulate bone material is the particulate material from the manufacturing method for a particulate bone material (100), which can have a particle size between 50 and 150 microns. On the other hand, the biopolymer can be PLA, however, other polymers known to a person ordinarily skilled in the art can be used for extrusion in a 3D printer, being bio-compatible and bio-absorbable with mechanical resistance suitable for bone implants (PLA, PLG or their mixtures are standard). Optionally, the biopolymer is PLA INGEO 2003D. Additionally, the ratios by volume can be between 60% and 80% biopolymer and between 40% and 20% bone particulate material respectively. On the other hand, the fact that the biopolymer is PLA prevents the extruder or the printing nozzle from clogging when using bone-polymer filaments in a 3D printer.

Optionally, regarding step b) of the method for manufacturing filament to make bone implants, the biopolymer and the particulate bone material are mixed in a container until a homogeneous mixture is obtained. In turn, and with respect to step c), the mixture of biopolymer and particulate bone material is heated to a temperature between 160° C. and 200° C. Optionally, the temperature to which it is heated is between 170° C. and 180° C. for a period of at least 3 minutes.

Additionally, the mixing of step b) can be carried out by mechanical means (e.g., mechanical mixers), wherein said mixers can have heating means, which allows the mixture to be heated to a temperature between 160° C. and 200° C., while being mixed. Optionally, heating when carried out while being mixed, is carried out for a time of at least 5 minutes, preferably between 3 minutes and 5 minutes.

Said step c), and given that the filament is a bone-biopolymer matrix, allows obtaining a homogeneous mass, which lets the filament to be extruded in different types of extruder machines. This also makes it possible to form different types of filaments with the required diameters.

For step d), the extrusion of the filament to make bone implants is preferably carried out using a single-screw or double-screw extruder machine having a nozzle with a diameter between 1.5 mm and 2.5 mm, preferably with a diameter 1.9 mm.

Said process is carried out at a temperature between 160° C. and 220° C., preferably at a temperature between 170° C. and 190° C. In addition, the extrusion process can be carried out at screw rotation speeds between 10 and 15 RPM. Preferably the process is carried out at a screw rotation speed of 12 RPM. The maximum extrusion torque can be between 50 Nm to 66.36 Nm, preferably at a temperature of 178° C. in the extrusion nozzle; additionally, the stabilization torque is 10.37 Nm at a temperature of 150° C. in the extrusion nozzle.

This allows obtaining a homogeneous filament (2000) that can be used in 3D printing, avoiding clogging of the injection nozzles or the extruder in 3D printing processes with a caliber corresponding to a 1.9 mm maximum diameter, although this can be 1.75 mm. For the understanding of the present invention, it shall be understood that the filament is a seamless bone-biopolymer filament, which does not break during the extrusion process. Additionally, said resulting filament is osseointegrable and regenerative.

Furthermore, said bone-biopolymer filament (2000) serves as a raw material for the manufacture of orthopedic implants, according to the method described above, e.g., by 3D printing, and it can also be used as a bone graft for filling in orthopedic and dental injuries, because the bone-biopolymer filament (2000) obtained melts in the presence of heat, allowing the molding of pieces with complex shapes.

On the other hand, in an embodiment of the invention to manufacture an implant by 3D printing with the bone-biopolymer filament (2000), a method for manufacturing implants by 3D printing (300) is carried out, which consists of the following steps:
designing the bone implant to be manufactured; and
modeling the bone implant in CAD software; and
processing the modeling in order to obtain an algorithm compatible with 3D printing software.

Wherein, the previous method allows obtaining parameters and the material (filament) required for the printing to be correct with the desired properties, and then the shape with the measurements of the plan comes out. On the other hand, the algorithm which can be obtained through the CAD software can be implemented in different types of software compatible with 3D printing machines, which allows configuring the printing parameters in order to obtain a print of the implant, according to the desired design. Additionally, said 3D printing can be made with a material that can be extruded and assimilated by the user's body wherein said implant will be placed, such as the bone-biopolymer filament (2000) produced through the method for manufacturing bone-biopolymer filament (200).

In one embodiment of the invention, the 3D printing temperature of a bone implant is between 180° C. to 230° C., preferably 200° C. to 210° C. On the other hand, the extruder nozzle of the 3D printing machine used to print a bone implant can be between 0.4 mm and 1.2 mm, preferably between 0.5 and 0.8 mm. Furthermore, the 3D printing speed can be between 20 mm/s and 85 mm/s, preferably between 30 mm/s and 50 mm/s.

Once a 3D printed implant for bone reconstruction (3000) is formed, said implant can be left with the following properties:
a surface roughness of 12.616±1.756 μm;
an implant hardness of 73.7±1.70 Shore D;
a flexural strength of 37.45±7.46 MPa;
a maximum bending stress of 69.2±13.68 Mpa; and
an implant elastic modulus of 1.49±0.17 Gpa.

One of the technical effects of the 3D printed bone reconstruction implant (3000) having the aforementioned features is to allow the 3D printed bone reconstruction implant (3000) to be fully osseointegrable and regenerative, i.e., the 3D printed bone reconstruction implant (3000) induces the generation of new bone within the patient's body. Additionally, the fact that said 3D printed bone reconstruction implant (3000) includes particulate bone material (1000), is that osseointegration and regeneration of the patient's injury is performed in less time, compared to other bone implants, e.g., polymeric, steel or titanium implants.

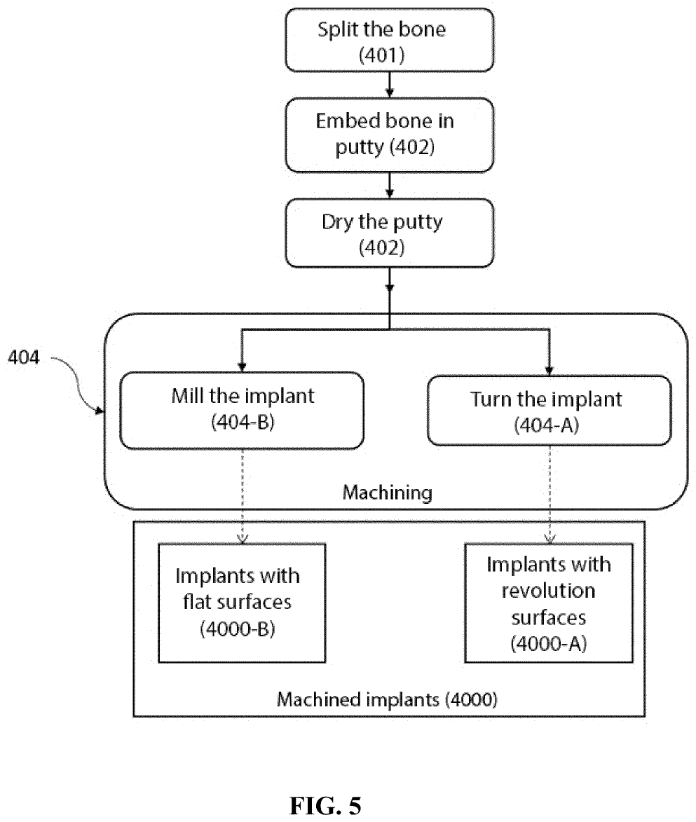

On the other hand, and with respect to the types of manufacturing initially named, the present invention also refers to a manufacturing method by machining, i.e., a set of mechanical operations for the shaping of parts by removing material by chip removal. Specifically, referring to FIG. 5, the present invention refers to a method for manufacturing bone implants by machining (400). Specifically, said method includes the following steps:
a) providing a bone material;
b) splitting the bone material;
c) encapsulating the split bone material of step b);
d) machining the encapsulated bone material in step c) to form an implant.

Where the machining can be turning the implant, milling the implant or a combination of both.

Specifically, and regarding step a) and step b), the split bone material may be bone, e.g., lyophilized (0000) cortical bone, preferably tibia and femur. Where, in order to make said cut, the bone is split longitudinally on the rough line from the proximal part to the distal part, forming bone sections with a length between 30 mm to 70 mm, preferably 45 mm, obtaining at least two equal bone sections in length and discarding the excess parts that may contain cancellous bone debris.

Optionally, each section of bone obtained can be radially cut every 30 degrees, as shown in the following diagram, obtaining 36 parts of bone, as shown in Chart 1.

Taking into account the high variation in dimensions and sizes of the bone, step b) corresponding to splitting the bone material according to Chart 1, thus letting take advantage of the greatest amount of material for use in the manufacturing method for bone implants by machining (400).

On the other hand, and with respect to step c) of the method for manufacturing bone implants by machining (400), corresponding to encapsulating the split bone material of step b), a mold is provided wherein it is filled with epoxy putty. Said putty does not contaminate the bone or generate contaminants when the implant is machined, since the putty is not liquid, which does not allow permeation of the porosity. The putty amount depends on the size of the bone and bone sections, because both the bone and the bone sections are not always uniform. Additionally, the putty amount required can be calculated by subtracting the volume of the mold with the volume of the bone that will be placed inside.

Subsequently, each of the bone material sections obtained in step b) is inserted into the mold with putty by pressure, e.g., by pressure exerted by a manual press or a hydraulic press. Additionally, the foregoing can optionally be carried out at low speeds because the bone can be fragmented if performed at high speeds. Once the bone sections are inserted into the mold with putty, excess putty can be removed if there is remaining putty. Then, the putty with the bone is left to dry for a period between 6 and 16 hours at a temperature between 15° C. and 30° C., in a particular example, the putty with the bone is left to dry for a period of time between 10 hours and 12 hours at a temperature between 20° C. and 25° C.

Once the putty with the bone sections is dried, a raw material with a shape determined by the mold is obtained, the mold is cut to be removed, thus obtaining a matrix of putty and freeze-dried bone that can be held in the different machines to be machined regardless of the dimensional variation that the bone material has had after step b) when it is split, either for curved or implants with flat surfaces.

The shape of the mold is selected from the group made up of cylinders, cubes, pyramids, prisms, equivalent shapes known by a person ordinarily skilled in the art or a combination of the above. Furthermore, the material of the mold is selected from the group made up of plastic, acrylic, wood, metal, equivalent materials known to a person ordinarily skilled in the art or a combination of the above. The fact that the mold has one of the previously mentioned shapes allows the putty with the bone sections to be easily removed from the mold, taking into account that acrylic and plastic have a lower coefficient of friction and are very low cost.

In one embodiment, the mold has a cylindrical shape, such as ½″ RDE 21 PVC tubes, wherein the putty and sections of bone material are inserted. Once the putty is dry, the mold is cut to extract the putty with the sections of the bone material with a curved shape, which allows it to be machined by turning and thus being able to obtain implants with a curved shape. In another modality, the mold can have some flat faces, which allows obtaining hardened putty with sections of bone material with a shape that can be machined by milling and thus obtaining implants with flat surfaces.

Now, regarding step d) of the method for manufacturing bone implants by machining (400) and corresponding to machining the bone material encapsulated in step c) to form a bone implant, it is decided which machining processes are required to manufacture the implants, i.e., if it requires external turning, milling, internal turning, facing, parting, grooving, internal threading, external threading, drilling, boring, reaming, knurling, or a combination thereof.

When it is required to form an implant with a cylindrical shape, the implant is turned as follows: a test tube of the putty is provided with the bone sections. Subsequently, a pressure-adjustable metal cup (Collet) is used, with a diameter similar to that obtained in the test tubes, which is used to mount the test tube and hold it in the mandrel of a lathe, in order to begin the turning process. Said turning can be carried out on any type of lathe, preferably on a CNC lathe, since this allows better control of the measurements of said machining and automates the manufacturing process. Then, some turning sub-steps are carried out corresponding to facing and turning the specimen until a cortical bone axis is obtained.

To carry out any machining according to step d) any cutting tool can be used, preferably Tungsten Carbide tools, coated with Titanium, Tantalum, Niobium, or a mixture thereof. It is recommended to use an insert reference ISO VNGG 16 04 12-SGF 1105 as a tool.

Moreover, the radius of the cutting tip can be any, preferably a radius ranging between 0 and 0.2 mm.

For facing, the cutting depth for each pass to face the specimen can be between 0.1 to 1 mm, preferably between 0.4 mm and 0.6 mm. On the other hand, the cutting tool feed can be between 0.1 and 0.3 m/min, preferably between 0.12 and 0.15 m/min. The speed of said machining is from 24 to 28 m/min. In a particular example, the speed is 25.45 m/min. Said facing is carried out until the length required for the implant is obtained.

However, once the specimen is faced, it is turned in order to obtain the diameter required for the implant. The speed of said turning is from 24 to 28 m/min. In a particular example, the speed is 25.45 m/min. The depth of each pass to rough down the material is between 0.1 to 1 mm; in a particular example, the depth of each pass to rough down the material is 0.6 mm. In turn, the cutting tool feed must be between 0.1 to 0.3 m/min. In one embodiment of the invention, the cutting tool feed is 0.15 m/min. The machining speed is from 24 to 28 m/min. Optionally, the machining speed is 25.45 m/min. These machining parameters are required to prevent the bone from calcination due to the machining temperature, as well as to prevent the fracture of the material due to the bending generated by the cutting tool on the material at the time of machining.

In an embodiment of the invention, the turning operation is carried out in sections of short lengths in the test tube, in order to prevent the bending caused by the cutting tool from causing fracture of the implant, wherein said sections can be between 100 mm or less. In order to understand the present invention, the test tube shall be understood as the combination of putty plus bone material encapsulated in step c) of the method for manufacturing bone implants by machining (400).

Additionally, in the event that the bone implant requires external thread (e.g., cortical screw) or internal thread, the implant is threaded with the required depths and measurements. The threading speed can be from 5 to 6 m/min, preferably 5.59 m/min. On the other hand, the depth of each pass to cut the implant material is preferably decreasing with a range from 0.1 mm to 0.001 mm. The foregoing, in order to avoid breaking the implant due to bending or breaking the thread crests. Furthermore, the advance of the cutting tool must be described by the thread pitch to be manufactured.

On the other hand, and taking into account that the machining processes described above are carried out on a dry material, preferably the use of cutting fluids in turning should be avoided, since this causes expansion of the material when it becomes wet, causing variation on the material dimensions and preventing the machining measurements from being those required. Except in threading, where a cutting fluid can be used, this fluid can be 0.9% saline solution. The cutting fluid can be applied in a spray, optionally between 1 to 5 ml of fluid per turned part, preferably 2 ml. If these amounts are exceeded, the material may show expansion due to hydration and the final measurements of the implant may change. Moreover, it can cause the machining to have undesired surface texture. Also, 90% alcohol can be used as a cutting fluid in the amounts deemed necessary, but it is important to emphasize that excess alcohol as a cutting fluid can agglomerate chips at the tip of the tool and affect the cutting process, generating material tearing surfaces instead of actual cutting surfaces. The optimal amount of alcohol as a cutting fluid can be less than 5 mL per pass and the tip of the cutting tool can optionally be cleaned with a textile material, preferably every 2 passes.

Now, and with respect to step c) of the method for manufacturing bone implants by machining (400), when it is required to form an implant with a surface other than a cylindrical form, milling is performed as follows: a test tube of the putty is provided with the bone sections obtained in step b) where said test tube preferably has a rectangular shape or the turned test tube as mentioned above. Subsequently, said test tube is clamped in a press or milling table. Any type of milling machine can be used for this machining, preferably a CNC milling machine, which allows automatization of the implant manufacturing process.

Then, the test tube is milled until the desired shape is obtained. Where the milling conditions can be the following: the machining speed can be between 20 to 22 m/min, preferably 20.73 m/min. The depth of each pass to cut the material is preferably between 0.1 to 1 mm, preferably 0.5 mm. On the other hand, the cutting tool feed can be between 0.1 and 0.3 m/min, preferably between 0.12 and 0.15 m/min. In addition, the use of cutting fluids in milling should preferably be avoided, or 90% alcohol should be implemented because, as previously mentioned, a dry material is being machined, the use of fluids in machining can hydrate the implant, causing a variation in its dimensions, and also, thus allowing the machining of the desired surface texture.

Additionally, any cutting tool can be used, such as Tungsten Carbide tools, coated with Titanium, Tantalum, Niobium, equivalent milling tools known to a person ordinarily skilled in the art, or a combination thereof. Both the diameter and type of the tool will depend on the shape desired to obtain on the implant.

With the machining conditions described above for manufacturing bone implants by machining (400), the implant is prevented from fracturing at any point of the milling. In the same way, there is no overheating of the material causing the bone calcination in the process. In addition, the fact that the implant can be machined as mentioned above, the implant can have the dimensions and shapes desired by a surgeon or by a person ordinarily skilled in the art, which provides greater versatility and creativity when arranging the implant in a user.

Now, once the steps of the manufacturing method for bone implants by machining (400) previously mentioned, corresponding to cutting a bone; encapsulating the cut bone; and machining the encapsulated bone, to form a machined implant (4000), a fabricated bone reconstruction implant is obtained. Where said implant is completely osseointegrable and regenerative and also induces the generation of new bone within the body of a user where the implant is placed.

Additionally, being a freeze-dried bone implant, osseointegration and regeneration of the patient's injury is performed in a shorter time compared to other bone implants. In addition, said implant can be used as a bone graft to fill in orthopedic and dental injuries, for specific applications where the presence of a bone graft with a defined shape is required for structural or support needs, and it can also be used in orthopedic or dental applications where clamping or support mechanisms are required.

On the other hand, and once a bone implant is obtained, the present invention also refers to a texturing (500) of an implant for bone reconstruction, whether it is a 3D printed implant (3000), a mechanized implant (4000), or any other type of implant made of bone. In order to understand the present invention, it will be understood that an implant is textured and its external surface changes.

When any of the implants has a curved shape, the implant receives a finishing pass in the lathe with the following conditions:
a cutting depth between 0.01 mm and 0.2 mm, preferably 0.1 mm;
a cutting tool feed can be between 0.1 and 0.3 m/min, preferably 0.15 m/min; and
a machining speed is 24 to 28 m/min, preferably 0.45 m/min.

Where, preferably said machining is carried out using a tool that complies with the ISO VNGG 16 04 12-SGF 1105 standard.

On the other hand, and when the implant has surfaces that are not curved surfaces, e.g., surfaces with flat shapes, a finishing pass is made in the milling machine with the following conditions:
a machining speed is between 20 to 22 m/min, preferably 20.73 m/min;
a cutting depth between 0.01 mm and 0.2 mm, preferably 0.1 mm; and
a cutting tool feed can be between 0.1 and 0.3 m/min, preferably 0.15 m/min; and

Wherein preferably said machining is performed with shank mills.

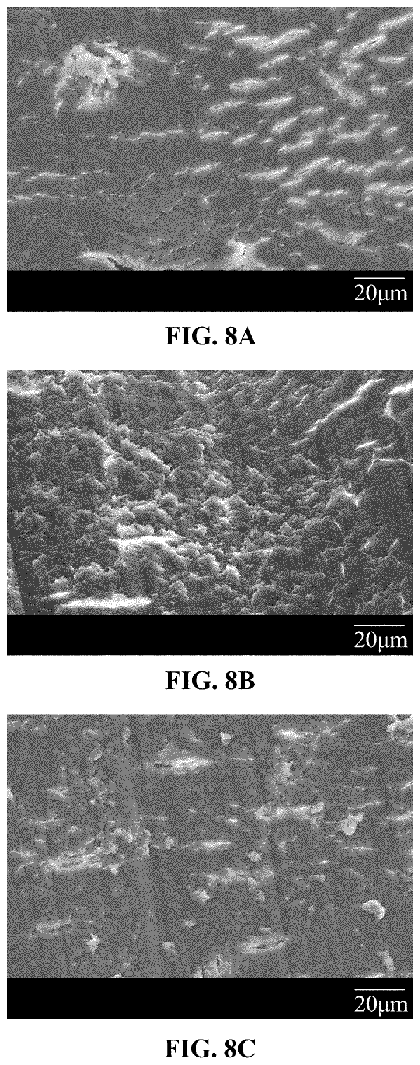

Said machining allows texturing the external surface of the bone implants, obtaining a surface roughness of: Ra=0.26 μm, with a deviation of 0.16 μm. For example, the lower limit of the roughness can be 0.10 μm, 0.2 μm, 0.15 μm, 0.25 μm or 0.13 μm. On the other hand, the upper limit of the roughness range can be 0.30 μm, 0.40 μm, 0.42 μm, 0.45 μm, 0.35 μm, 40 μm. Where said surface texture improves its osseointegration, decreasing the probability of implant rejection by the patient. Regarding FIG. 8, it shows two examples of implant texturing.

### Example 1

A pre-treatment method (000) was carried out on a bone material which included the steps of:
providing a bone material corresponding to a frozen bovine bone, specifically the tibia and femur;
removing all the remaining muscle tissue that exists by means of a blade until a bone is obtained;
cutting the bone into three pieces, to separate the part that has the compact bone (cortical) and discard the heads, which have cancellous bone;
soaking the bone material in water with detergent for 24 hours;
cleaning with the help of a blade and a brush, thoroughly scraping all the inside and outside of the bone;
soaking the bone material in 96% ethyl alcohol for 24 hours, in order to eliminate fat and blood residues;
repeating the steps of soaking the bone material in detergent water, cleaning and soaking the bone material in ethyl alcohol, three times;
freezing the bone pieces at a temperature of −50° C.; and
lyophilizing the bone in a vacuum pressure cabinet at 0.01 mbar, for 24 hours.

### Example 2

A bone implant was made using a bone implant manufacturing method by machining (400), for the manufacture of screw-type implants for cortical bone (HA) of reference HA 2.7×15 mm long. Where the manufacturing method for bone implants by machining (400) in this case included the steps of:
providing a lyophilized bone (0000), corresponding to two demineralized bovine bones, specifically a cut and lyophilized tibia and femur;
cutting the lyophilized bone (0000) into 42 mm lengthwise sections;
splitting and cutting the pieces of demineralized bone material from the previous step, radially, with a circular matrix every 30 degrees as shown in Chart 1, obtaining a total of 36 sections for each bone used;
encapsulating the sections of bone material from the previous step, wherein said encapsulation was made with a two-component epoxy putty and in a mold which had the following configuration: a tube with a diameter of half an inch by 42 mm long, wherein the encapsulation putty was left to dry in 12 hours at a temperature of 22° C. Wherein, once the putty had dried, the mold was removed by cutting it longitudinally, obtaining test tubes ready for machining.
machining the material test tube from the previous step to form the HA 2.7×15 mm screw-type implants, which comply with the dimension and tolerance specifications of the ISO 5835:1991 standard.

Additionally, two types of machining were made, a turning and a milling, with the following features:

The turning was made with a Sandvik Coromant tool reference ISO VNGG 16 04 12-SGF 1105, under the following conditions:

The test tube was turned and faced, until obtaining an axis of bone material only, which had the following features:
a cutting depth of 0.6 mm;
a 0.15 m/min cutting tool feed; and
a machining speed of 25.45 m/min.

For internal turning the bone, until obtaining the HA 2.7×15 mm implant shape:
a cutting depth of 0.6 mm;
a 0.15 m/min cutting tool feed; and
a machining speed of 25.45 m/min.

The internal turning and shape of the implant was machined in three 7.5 mm sections, two to comply with the 15 mm screw length of the implant, then the implant head was turned and formed in the last section.

On the other hand, the threading of the implant, until obtaining the required thread in the HA 2.7×15 mm screw-type implant, was done with the following machining parameters:
a decreasing cutting depth in each pass from 0.1 mm to 0.001 mm;
a cutting tool feed of 1 mm per thread (thread pitch); and
a machining speed preferably 5.59 m/min.

Where, the cutting tool was a high-speed steel turning tool at 12% Co, sharpened with the angles described in section 4.1 of the ISO 5835:1991 standard, for HA 2.7 compact bone orthopedic screws.

On the other hand, for milling the hexagonal head of the screw-type implant was made, the machining was carried out with the following conditions:
a machining speed of 20.73 m/min;
a cutting depth of 0.5 mm; and
a 0.15 m/min tool feed.

Where, the tool was a 6 mm HSS shank milling cutter.

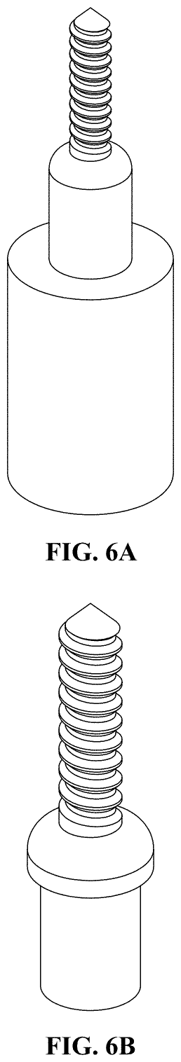

Additionally, and regarding FIG. 6A, the bone implant is shown wherein one of its ends has a part of the thread machined, while the other end has a larger diameter corresponding to the area that was arranged on the lathe to carry out the turning. In turn, FIG. 6B illustrates the implant formed from the method previously described, wherein it is already complete.

### Example 3

An implant texturing of the EXAMPLE 2, method (500), before it was threaded to ensure the surface finish in the crests of the screw, the tip and in the head. Wherein, said texturing had the following machining features:

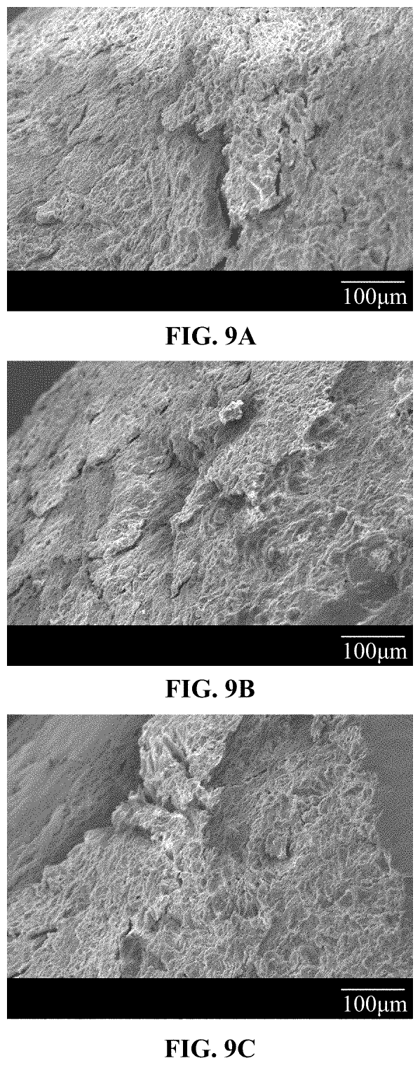

In the HA 2.7×15 mm screw-type implant in its cylindrical parts, texturing turning was performed with the following features:
a cutting depth of 0.1 mm;
a 0.15 m/min cutting tool feed; and
a machining speed of 25.45 m/min.

Where, the tool was a Sandvik Coromant reference ISO VNGG 16 04 12-SGF 1105.

Wherein said implant was left with an average surface roughness that had the following features: A value Ra=0.26 μm with a deviation of sd=0.16 μm. Regarding FIGS. 8A, 8B, 8C, the surface finish at the bottom of the implants thread is illustrated. On the other hand, and regarding FIGS. 9A, 9B, 9C the surface finish on the radius of the implants head is illustrated.

### Example 4

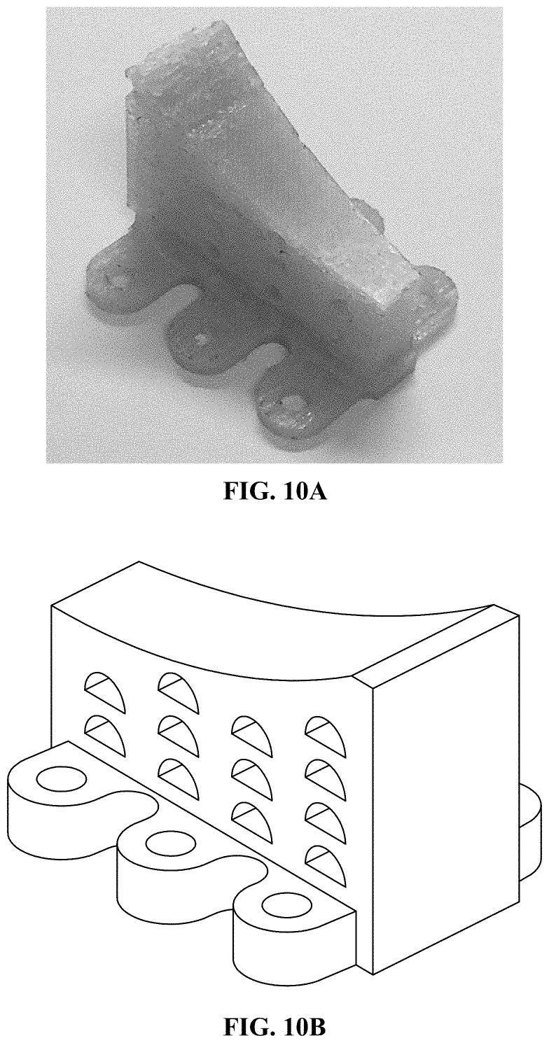

Regarding FIG. 10A and FIG. 10B, four bone implants were printed on a 3D printer and machined as illustrated in said figures.

Below are the roughness data obtained from the four implants manufactured:

TABLE 1
Roughness of printed bone implants.

| | Basis | Diagonal face | Rear face |
|---|---|---|---|
| Part | [μm] | [μm] | [μm] |
| 1. | 0.755 | 3.673 | 3.588 |
| 2. | 0.655 | 2.066 | 5.135 |
| 3. | 0.775 | 2.373 | 3.783 |
| 4. | 0.678 | 3.839 | 4.612 |
| Average | 0.716 | 2.988 | 4.280 |
| St. dev. | 0.050 | 0.778 | 0.626 |

### Example 5

A bone powder manufacturing method was carried out, which included two steps, a pre-milling step, where a coarse powder was obtained, and a pulverization step. The pre-milling step had the following steps:
providing a lyophilized bone (0000), corresponding to two demineralized bovine bones, specifically a cut and lyophilized tibia and femur;
cutting the freeze-dried bone (0000) into sections;
encapsulating the sections of lyophilized bone (0000) from the previous step, wherein said encapsulation was made with sugar candy and in a mold. Said mold had a cylindrical shape with a diameter of 10.16 cm by 30 cm long, two rubber caps to hold the test tube in a lathe, and a threaded rod central axis, wherein the caramel was left to dry for 24 hours at a temperature of 18° C. Once the caramel was dry, the mold was removed by cutting it longitudinally, thus obtaining test tubes ready for machining;
machining the material specimen from the previous step; and
separating the caramel chip and bone by a filter separation process, wherein the caramel was dissolved using warm water at 40° C. The bone powder was separated using a sieve to remove the sugar water mixture.

Wherein the machining was a turning made with the following features:
a cutting depth of 2 mm;
a 0.25 m/min cutting tool feed; and
a machining speed of 3.4 m/min.

In the machining step, the lathe was prepared with a collection box for particulate material and inlet isolation from any foreign body. The caramel and bone test tube is placed in the lathe and the piece is machined, obtaining with this process a coarse bone powder mixed with caramel.

Additionally, with the bone separated from the mixture, the bone was submerged in 96% ethyl alcohol for 6 hours and placed in a container that allowed the bone to dry under natural conditions for 3 days. Once the initial bone powder described above was obtained, a particle size of less than 200 and a form factor (FF) between 0.5 and 1 were obtained, using a high energy mill wherein about 20 to 30 grams of bone powder were deposited. Where said bone powder was pulverized for a time of 2:30 minutes to obtain the desired size and form factor (FF), and avoid bone powder overheating.

### Example 6

A bone-biopolymer filament was made with the following features: pulverized bone material with a particle size less than 200 μm. Additionally, PLA pellets were used as polymer material. Subsequently, said materials were mixed in weight ratio 80% PLA/20% bone powder and 95% PLA/5% bone powder, and combined in a hermetically sealed box and manual mixing was carried out by shaking the box.

Subsequently, said mixture was deposited in an internal Brabender double-screw mixer which was pre-heated with an ascending temperature profile as follows: 155° C., 160° C., 170° C., 170° C., 170° C., 170° C.

Once the filament was arranged in said machine, and with the machine ready, the filament was extruded. For this purpose, a water cooling platform was located at the exit of the extruder, from the opposite end the material was pulled by means of a calender that rotated at a speed of 21 RPM, thus winding the filament produced. The extruded filament under these parameters had a diameter of 1.65 mm with a standard deviation of 0.125 mm, wherein said diameter is optimal for fused deposition modeling (FDM).

### Example 7

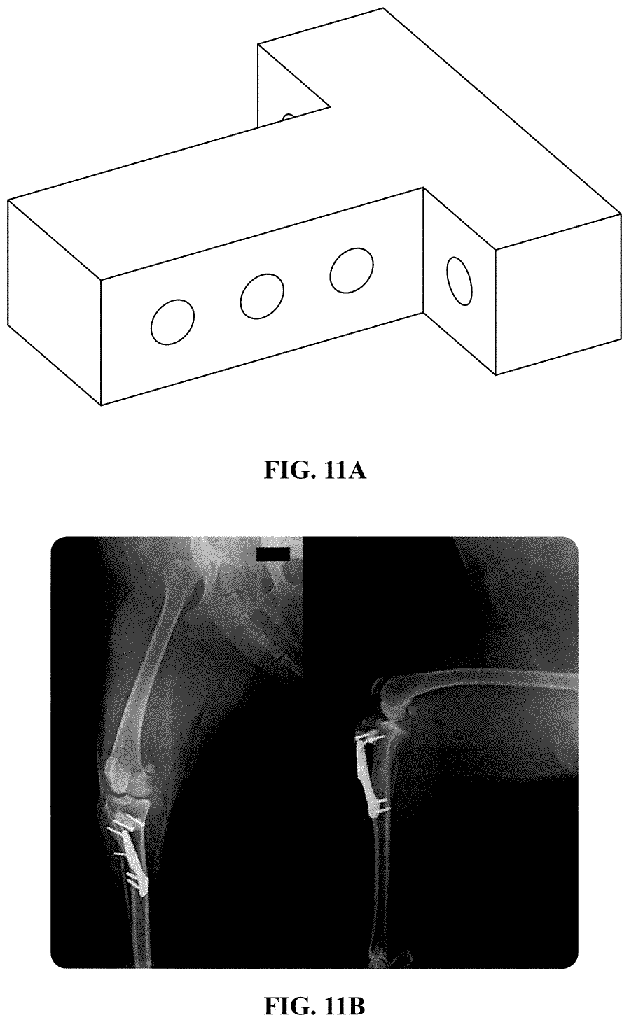

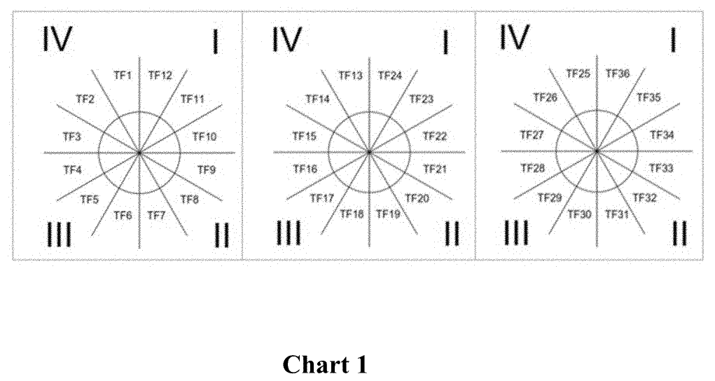

A method for manufacturing bone implants by machining (400) was carried out for the manufacture of TTA box-type implants. The manufacturing method for bone implants by machining (400) included the following steps:
a) providing a lyophilized bone (0000); corresponding to two demineralized bovine bones, specifically a cut and lyophilized tibia and femur;
b) cutting the aforementioned bone material into 42 mm longitudinal sections.
c) encapsulating the sections of bone material from the previous step, wherein said encapsulation was made with a two-component epoxy putty and in a mold which had the following configuration: a width of 50 mm by a length of 50 mm, by a thickness of 25 mm. Wherein the encapsulation putty was allowed to dry in a time of 12 hours, at a temperature of 22° C.;
d) removing the mold once the putty has dried, cutting it longitudinally with one, obtaining the rectangular test tubes ready for machining; and
e) machining the material test tube from the previous step to form the TTA box-type implants, which met the dimension specifications of 18.8 mm wide, 20.6 mm high and 9.8 mm wide according to FIG. 11A.

Wherein, the machining was carried out through a milling process, with the following features:
a machining speed of 20.73 m/min; and
a 0.15 m/min tool feed;

Where, the tool was a 6 mm high speed steel shank milling cutter.

Additionally, two 2.2 mm through holes were made at the ends of the base and three 3 mm through holes along the body of the implant. These holes were made with the following machining features:
a machining speed (drilling) of 20.73 m/min; and
a 0.15 m/min tool feed.

Where, the tool was a 3 mm and 2.2 mm high speed steel shank milling cutter, respectively.

This TTA type implant was tested in Tibial Tuberosity Advancement (TTA) surgery in a dog. FIG. 11b below, the x-ray immediately after surgery, wherein the patient recovered without problem 45 days after surgery.

### Example 8

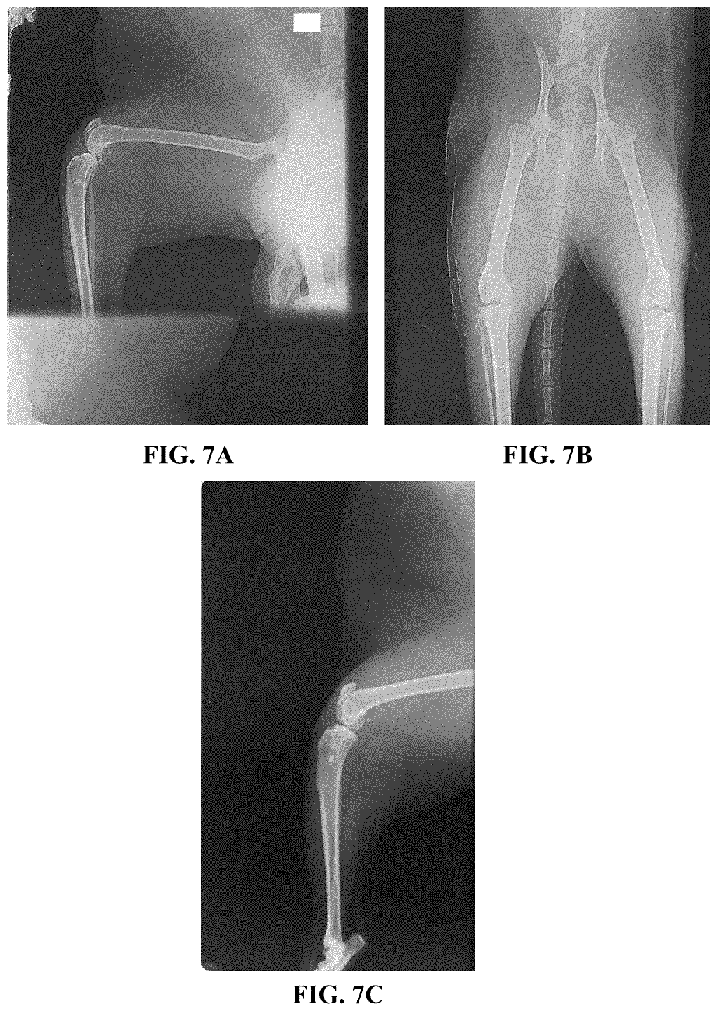

Regarding FIGS. 7A, 7B and 7C, 3 x-rays of a machined bone implant are illustrated, wherein FIG. 7A shows the implant immediately after the surgery where it was implanted. FIG. 7B shows surgery 30 after said implant was implanted.

It shall be understood that the present invention is not limited to the modalities described and illustrated because, as will be evident to a person versed in the art, there are possible variations and modifications that do not depart from the spirit of the invention, which is only defined by the following claims.

1. A method for texturing a bone implant (500), comprising:
providing a bone implant; and
machining the bone implant by means of a cutting tool;
wherein the machining has the following features:
a cutting depth between 0.1 mm and 2 mm;
a tool feed between 0.1 to 0.3 m/min;
a cutting speed between 24 to 28 m/min for turning operations; and
a cutting speed between 20 to 22 m/min for milling operations.

2. The method of claim 1, wherein the tool feed is between 0.1 to 0.3 m/min for machining by milling.

3. The method of claim 1, wherein the cutting speed for turning operations is 24 to 28 m/min for machining by turning.

4. The method of claim 1, wherein the speed for turning operations is 20.73 m/min for milling operations.

5. The method of claim 1, wherein the cutting tool is selected according to the ISO 1832 VNGG 16 04 12-SGF 1105 code for turning.

6. The method of claim 1, wherein the cutting tool is a shank mill for milling.

7. A method for manufacturing bone implant (400), comprising:
i) providing a bone material;
ii) splitting the bone material into sizes between 30 mm to 70 mm;
iii) encapsulating the split bone material of step b) in a mold with putty;
iv) machining the bone material encapsulated in step c) by means of a cutting tool to form a bone implant.

8. The method of claim 7, wherein the machining parameters of step iv) are:
a cutting depth between 0.1 and 1 mm;
a tool feed between 0.1 and 0.3 m/min; and
a cutting speed between 24 to 28 m/min;
wherein, when the machining is a turning, it has the following parameters:
a cutting depth between 0.1 and 1 mm;
a cutting tool feed between 0.1 to 0.3 m/min; and
a machining speed between 24 to 28 m/min;
wherein, when the machining is a milling, it has the following parameters:
a cutting depth between 0.1 and 1 mm;
a cutting tool feed between 0.1 to 0.3 m/min; and
a machining speed between 20 to 22 m/min.

9. The method of claim 8, wherein the cutting tool is selected according to the ISO code VNGG 16 04 12-SGF 1105 for turning.

10. The method of claim 8, wherein the cutting tool for milling is a shank mill.

11. The method of claim 8, wherein the machining has a cutting depth between 0.1 mm and 1 mm.

12. The method of claim 8, wherein the cutting tool feed is between 1 to 0.3 m/min in turning.

13. The method of claim 8, wherein the machining speed is between 24 to 28 m/min in turning.

14. The method of claim 8, wherein a thread is made to the encapsulated implant material of step c) in turning, with the following machining parameters:
i) a threading speed between 5 to 6 m/min; and
ii) a pass depth for cutting the material is decreasing from 0.1 mm to 0.001 mm.

15. A method for manufacturing a particulate bone material (100), comprising:
a) providing a bone material;
b) splitting bone material into fragments;
c) embedding the split bone material in a soluble containment matrix;
d) drying the soluble containment matrix until a solidified containment matrix is obtained;
e) machining the solidified containment matrix of step d) by means of a cutting tool to obtain particulate bone material plus containment matrix; and
f) removing the particulate bone material from the containment matrix.

16. The method of claim 15, wherein the machining is a turning that has the following features:
1. a cutting tool feed between 0.01 mm/rev and 0.06 mm/rev;
2. a cutting speed between 3 and 4 m/min;
3. a rake angle between 3° and 5°;
4. a clearance angle between 5° and 10°;
5. a main steering angle between 50° and 65°; and
6. a cutting depth between 0.02 and 0.05 mm.

17. The method of claim 15, where a high speed steel turning tool at 12% Co is used in the turning process.

18. The method of claim 15, wherein the containment matrix is molten sucralose.

19. The method of claim 15, wherein the particulate bone material is removed from the containment matrix with water at a temperature between 20° C. and 30° C.

20. The method of claim 15, wherein the particulate bone material obtained has a size between 90 μm and 120 μm.

21. The method of claim 15, wherein the particulate bone material obtained has an form factor (FF) between 0.5 and 1.

22. A method for manufacturing a bone implant, comprising:
a) providing particulate bone material with a particle size between 90 μm and 120 μm and form factor (FF) between 0.5 and 1;
b) mixing the particulate bone material with a biopolymer; and
c) obtaining filaments by means of an extrusion process;
d) forming an implant using a 3D printer with the following features:
an extrusion temperature between 160° C. and 230° C.;
a screw rotation speed between 10 and 15 RPM;
an extrusion Torque between 50 Nm and 70 Nm;
a stabilization torque between 8 Nm and 12 Nm at a temperature between 130° C. and 160° C. in the extrusion nozzle; and
a printing speed is between 20 mm/s to 85 mm/s. Preferably 50 mm/s.

23. The method of claim 22 wherein a bone implant is texturized by means of machining, where the machining has the following features:
a cutting depth between 0.1 mm and 2 mm;
a tool feed between 0.1 to 0.3 m/min;
a speed between 24 to 28 m/min for turning operations; and
a speed between 20 to 22 m/min for milling operations.

24. A bone implant comprising a surface roughness between 0.10 μm and 40 μm.

25. A particulate bone material (1000) with a diameter between 50 μm and 250 μm, and form factor (FF) between 0.5 and 1.

26. The particulate bone (1000) of claim 25, which also has a diameter between 90 μm and 120 μm.
